(19) United States
(12) Patent Application Publication
(10) Pub. No.: US 2026/0123614 A1
(43) Pub. Date: May 7, 2026

(54) HEAT-TOLERANT SOFT-SHELL CLAMS

(71) Applicant: MORGAN STATE UNIVERSITY, Baltimore, MD (US)

(72) Inventors: Ming Liu, St. Leonard, MD (US); Brittany Wolfe-Bryant, St. Leonard, MD (US); Jon Farrington, St. Leonard, MD (US)

(21) Appl. No.: 19/380,181

(22) Filed: Nov. 5, 2025

Related U.S. Application Data
(60) Provisional application No. 63/716,465, filed on Nov. 5, 2024.

(51) Int. Cl. A01K 67/30; C12Q 1/6888

(52) U.S. Cl. CPC A01K 67/30; C12Q 1/6888; C12Q 2600/124

(57) ABSTRACT

This invention relates to the development of heat-tolerant soft-shell clam (Mya arenaria) lines for aquaculture through selective breeding and molecular genetic approaches. Two methods are employed: (1) phenotype-based selection, in which clams are exposed to natural or controlled heat shock conditions (30-33° C.) and survivors are selected across successive generations and multiple populations; and (2) marker-assisted selection (MAS), utilizing single nucleotide polymorphisms (SNPs) identified through genome-wide association studies (GWAS) to select broodstock capable of producing heat-tolerant offspring. The resulting lines exhibit enhanced survival and endurance under elevated temperatures, particularly above the species' critical threshold of 28° C. These heat-tolerant clams enable sustainable aquaculture production in Maryland and other warm, low-salinity estuaries where soft-shell clams historically failed to survive summer heat.

### GOVERNMENT RIGHTS

This invention was made with government support under Maryland Sea Grant Award number NA180AR4170070 awarded by the National Oceanographic and Atmospheric Administration. The government has certain rights in the invention.

### FIELD OF THE INVENTION

This invention relates to aquaculture, specifically the breeding and propagation of heat-tolerant clams.

### BACKGROUND OF THE INVENTION

Maryland's current aquaculture industry is primarily focused on a single species, the Eastern oyster, because the major aquaculture leases are located in the upper and middle Chesapeake Bay where salinity is low. Other popular seafood species struggle to survive in these low salinity environments. This monoculture leaves the aquaculture industry vulnerable to disease, climate change, and market fluctuations. The soft-shell clam (Mya arenaria, hereafter Mya) was once a major component of Maryland's shellfish fishery and is naturally adapted to low-salinity waters (<10 ppt). However, due to a combination of climate change, overfishing, disease, and predation, landings have declined to a small fraction of their historical levels. This decline has motivated growers to explore Mya as an alternative aquaculture species to diversify production and enhance business resilience. Leveraging the shellfish breeding program established at Morgan State University (MSU) Patuxent Environmental and Aquatic Research Laboratory (PEARL), the team successfully produced Mya seed that reached an average shell length of approximately 1.8 inches within eight months under multiple culture methods. Yet, a critical challenge remains: farmed clams fail to survive Maryland's summer, when peak temperatures exceed the species' lethal threshold (˜28° C.). As Mya lies near its southern distribution limit, natural behaviors such as retreating to deeper, cooler sediments are difficult to replicate in culture systems. Previous heat-shock experiments, however, revealed substantial variation in survival among individuals (FIG. 1), suggesting that selective breeding could enhance heat tolerance and enable sustainable Mya aquaculture in Maryland.

### SUMMARY OF THE INVENTION

This invention involves the development of heat-tolerant soft-shell clam lines using two selective breeding approaches. The first approach, phenotype-based selection, involves exposing clams to controlled or natural heat shock conditions and selecting survivors across multiple generations. The second approach, marker-assisted selection (MAS), utilizes genetic markers associated with heat tolerance to identify superior broodstock. The resulting heat-tolerant clam lines exhibit enhanced survival and endurance at elevated temperatures, particularly under the critical thermal threshold of 28° C., enabling improved aquaculture performance in warm and variable environments, which is critical to farming success in Maryland. It is specifically noted that every combination and sub-combination of the above-listed and below-described features and embodiments is considered part of the invention.

### BRIEF DESCRIPTION OF DRAWINGS

The foregoing summary, as well as the following detailed description of the preferred invention, will be better understood when read in conjunction with the appended drawings. For the purposes of illustrating the invention, there are shown in the drawings embodiments which are presently preferred. It should be understood, however, that the invention is not limited to the precise arrangements and instrumentalities shown. In the drawings:

FIG. 1 is a graph showing Daily Mortality of Soft-Shell Clams in the indoor heat shock experiment conducted in 2025.

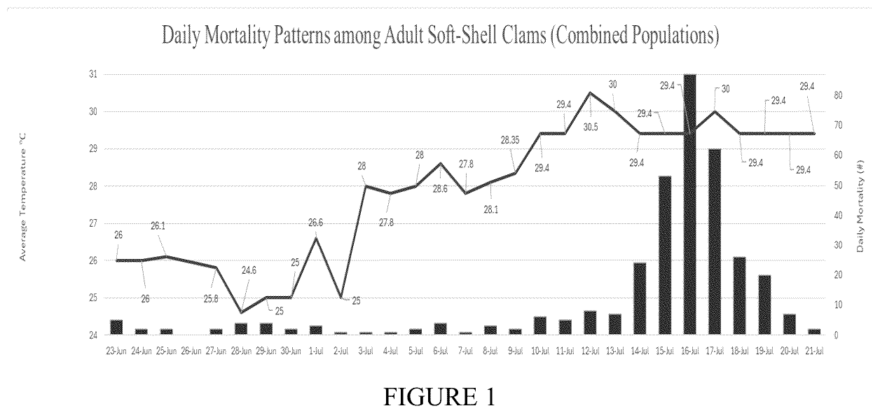

FIG. 2 is a chart showing average overall mortality of juvenile soft-shell clams based on F1 HT2023 (labeled as HTF1 in this figure below) seed lines vs. wild-type seeds.

### DETAILED DESCRIPTION OF THE INVENTION

Method (1): phenotype-based selection.

Step (a): First selection for heat tolerant Mya through heat shock challenge.

In the summer of 2023, approximately 5,000 seed from the line MD040623 were deployed in PEARL's pier with ambient seawater for the entire summer. The clams experienced continuous heatwaves in July, with the highest recorded seawater temperature reaching 33° C. By the end of the heatwave, 261 clams remained after weekly checks, when the water temperature had stabilized below 28° C. These survivors were preserved as the first heat-tolerant clam line and designated HT2023.

In the summer of 2024, the selection was repeated using the line produced in spring 2024, designated MD32824. A total of 179 clams survived after the water temperature declined below 28° C. in September. These survivors were preserved as the second heat-tolerant clam line, designated HT2024.

Second-round selection for F1 Heat-Tolerant Mya Line:

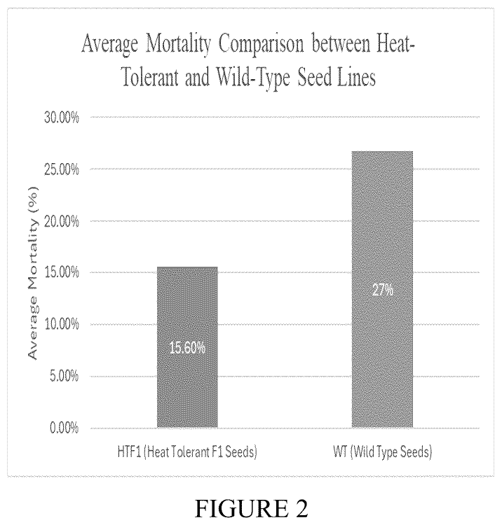

In spring 2025, the team conditioned the two-year-old HT2023 clams, and successfully produced the F1 HT2023 generation. A preliminary evaluation comparing the F1 HT2023 line (designated HT2023F1) with a control line bred from wild broodstock under summer heat conditions showed 11.4% higher survival in the HT2023F1 line, see FIG. 2, demonstrating promising improvement in heat tolerance.

All HT2023F1 and control line seed were cultured at the PEARL pier under ambient seawater conditions throughout the summer. Survivors from each line were preserved and designated as HT2023F1 and HT2025, respectively. The HT2023F1 line underwent two rounds of selection, whereas the HT2025 line experienced a single round of selection. These survivors from the 2025 selection will be maintained and reared for a subsequent round of selection during the summer of 2026, ensuring continued genetic improvement and advancement of heat-tolerant traits.

Method (2): Marker-Assisted Selection.

In some bivalve species, heat tolerance has been found to exhibit moderate to high heritability (0.3-0.5), indicating that the trait may be controlled by, or associated with, a limited number of genes. Accordingly, significant single nucleotide polymorphism (SNP) markers may be identified and utilized to enable rapid selection through marker-assisted selection (MAS).

Step (a): Heat shock experiment to construct training populations.

In the summer of 2024, an indoor heat shock experiment was conducted at the hatchery to evaluate differences in heat tolerance under varying rearing conditions and to establish training populations for genome-wide association analysis (GWAS) aimed at identifying genetic markers. Clams were reared under three conditions: (i) exposed (“naked”), (ii) enclosed within a “sandwich” type net gear, and (iii) individually in sand-filled bottles. Seawater temperature was gradually increased from 26° C. and maintained at 30° C. Mortality was monitored twice daily; deceased clams were collected and preserved in 95% ethanol for genomic DNA extraction, with shell length recorded and date-of-death documented as the heat tolerance phenotype. Results indicated that clams reared in sand exhibited greater heat tolerance than those reared in naked or sandwich-like environments, with lethal temperature thresholds approximately 1° C. higher than sand-free groups. After three weeks of exposure, only 19 clams from sand-filled bottles and one clam from the naked environment survived. A total of 783 dead samples were preserved.

In the summer of 2025, a complementary heat shock experiment was conducted at the PEARL hatchery to expand the sample size for genome-wide association study (GWAS) analysis. A total of 346 adult clams were collected from three geographic sites—Poplar Island, Honga River, and St. Leonard Creek—and were raised using ambient seawater. The water temperature gradually increased from 26° C. to 30° C., following a similar protocol to the 2023 experiment. All deceased clams were collected, preserved in 95% ethanol, and their date of death recorded as the phenotypic indicator of heat tolerance.

Notwithstanding the specific embodiments, features, elements, combinations and sub-combinations disclosed herein, it is expressly considered and here disclosed that every single element, every single feature, and every combination and sub-combination thereof disclosed herein may be combined with every other element, feature, combination and sub-combination disclosed herein.

It will be appreciated by those skilled in the art that changes could be made to the embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A heat-tolerant soft-shell clam (Mya arenaria) line developed from Maryland wild populations at the Morgan State University Patuxent Environmental and Aquatic Research Laboratory (MSU PEARL), wherein the line is genetically distinct from unselected wild populations and characterized by enhanced survival at seawater temperatures exceeding 28° C., and wherein the line is produced by one or more of the following:
(a) phenotype-based selection under controlled or ambient heat challenge, and/or
(b) marker-assisted selection (MAS) based on genetic markers associated with heat tolerance.

2. The clam line of claim 1, wherein the line is produced through phenotype-based selection by exposing hatchery seed or juveniles to natural heatwaves with seawater temperatures between 30° C. and 33° C. during the summers of 2023, 2024, and 2025, and preserving surviving individuals for subsequent breeding, the resulting lines being designated HT2023, HT2024, and HT2025.

3. The clam line of claim 2, wherein survivors from a heat-shock selection conducted on the F₁ population bred from the HT2023 line in Summer 2025 are designated HT2023F₁.

4. The clam line of claim 3, wherein the HT2023F₁ line demonstrates approximately 11.4% greater survival compared to an unselected control line under equivalent thermal stress conditions.

5. The clam line of claim 1, wherein marker-assisted selection (MAS) is employed using a panel of single nucleotide polymorphisms (SNPs) significantly associated with heat tolerance, identified through double-digest restriction-site associated DNA sequencing (ddRAD-seq) and genome-wide association studies (GWAS).

6. The clam line of claim 5, wherein the identified SNP panel is used to genotype at least 300 broodstock, and breeding values are estimated to select the top 10% of individuals as heat-tolerant broodstock and the bottom 10% as less-tolerant controls.
